(19) United States
(12) Patent Application Publication
(10) Pub. No.: US 2025/0222265 A1
(43) Pub. Date: Jul. 10, 2025

(54) PATIENT INTERVENTION OF ANTITACHYARRHYTHMIA THERAPY

(71) Applicant: Medtronic, Inc., Minneapolis, MN (US)

(72) Inventors: Matthew J. Hoffman, St. Paul, MN (US); Vladimir P. Nikolski, San Diego, CA (US); Aaron M. Saikin, Littleton, CO (US); Brad C. Tischendorf, Minneapolis, MN (US); Bridget A. Cavanagh, White Bear Township, MN (US)

(21) Appl. No.: 18/851,480

(22) PCT Filed: Mar. 30, 2023
(86) PCT No.: PCT/US2023/016954

§ 371 Date: Sep. 26, 2024

Related U.S. Application Data
(60) Provisional application No. 63/362,300, filed on Mar. 31, 2022.

(51) Int. Cl. A61N 1/39; A61N 1/372; G16H 20/40

(52) U.S. Cl. CPC A61N 1/39622; A61N 1/37217; A61N 1/37247; A61N 1/3925; G16H 20/40

(57) ABSTRACT

A medical system includes an implantable medical device (IMD) configured to detect an arrhythmia in a patient; and an external device comprising processing circuitry and configured to receive from the IMD an indication that the IMD has detected the arrhythmia in die patient; generate an output indicating that the IMD has detected the arrhythmia; receive an input from a user; and in response to the input from the user, transmitting to the IMD a command to cause the IMD to modify a therapy plan determined by the IMD.

This application claims the benefit of U.S. Provisional Patent Application No. 63/362,300, filed 31 Mar. 2022, the entire contents of which is incorporated herein by reference.

### FIELD

This disclosure generally relates to medical devices and, more particularly, to techniques and devices for delivering antitachyarrhythmia therapy to a patient.

### BACKGROUND

Malignant tachyarrhythmia, for example, ventricular fibrillation, is an uncoordinated contraction of the cardiac muscle of the ventricles in the heart, and is the most commonly identified arrhythmia in cardiac arrest patients. If this arrhythmia continues for more than a few seconds, it may result in cardiogenic shock and cessation of effective blood circulation. Consequently, sudden cardiac death (SCD) may result in a matter of minutes.

In patients with a high risk of ventricular fibrillation, the use of an implantable medical device (IMD), such as an implantable cardioverter defibrillator (ICD), has been shown to be beneficial at preventing SCD. An ICD is a battery powered electrical shock device, that may include an electrical housing electrode (sometimes referred to as a can electrode), that is typically coupled to one or more leads having one or more additional electrodes. The lead electrodes may be placed within the heart, or outside the heart, e.g., subcutaneously or substernally. If an arrhythmia is sensed, the ICD may deliver an antitachyarrhythmia shock, e.g., a cardioversion or defibrillation shocks, to shock the heart and restore its normal rhythm. Some ICDs have been configured to attempt to terminate detected tachyarrhythmias by delivery of anti-tachycardia pacing (ATP) prior to delivery of a shock. Additionally, ICDs have been configured to deliver relatively high magnitude post-shock pacing after successful termination of a tachyarrhythmia with a shock, in order to support the heart as it recovers from the shock. Some ICDs also deliver bradycardia pacing, cardiac resynchronization therapy (CRT), or other forms of pacing.

### SUMMARY

According to one example, a method includes receiving, at an external device from an implantable medical device (IMD) implanted in a patient, an indication that the IMD has detected an arrhythmia in the patient; generating, by the external device, an output indicating that the IMD has detected the arrhythmia; receiving, at the external device, an input from a user of the external device; and in response to the input from the user, transmitting, from the external device to the IMD, a command to cause the IMD to modify a therapy plan determined by the IMD.

According to another example, a device includes a memory; and processing circuitry coupled to the memory and configured to: receive, from an implantable medical device (IMD) implanted in a patient, an indication that the IMD has detected an arrhythmia in the patient; generate an output indicating that the IMD has detected the arrhythmia; receive, at the external device, an input from a user of the external device; in response to the input from the user, transmit to the IMD a command to cause the IMD to modify a therapy plan determined by the IMD.

According to another example, a system includes an implantable medical device (IMD) configured to detect an arrhythmia in a patient; and an external device comprising processing circuitry and configured to: receive from the IMD an indication that the IMD has detected the arrhythmia in the patient; generate an output indicating that the IMD has detected the arrhythmia; receive an input from a user; in response to the input from the user, transmitting to the IMD a command to cause the IMD to modify a therapy plan determined by the IMD.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example system for predicting and treating cardiac arrhythmia in accordance with the techniques of the disclosure.

FIG. 2 is a front view of a patient implanted with an extracardiovascular ICD system implanted intra-thoracically.

FIG. 3 is a block diagram illustrating an example configuration of the implantable medical device (IMD) of FIG. 1.

FIG. 4A is a functional block diagram illustrating an example configuration of the external device of FIG. 1.

FIGS. 4B and 4C show examples of the external device in FIG. 4A.

FIG. 5 shows a flow diagram of a process that may be performed by an IMD and an external computing device.

FIG. 6 shows a flow diagram of a process that may be performed by an IMD without an external computing device.

FIG. 7 shows an example device that may be used in conjunction with the techniques of FIG. 6.

Like reference characters refer to like elements throughout the figures and description.

### DETAILED DESCRIPTION

A significant proportion of treated tachy-arrhythmias are due to supraventricular tachycardia, sinus tachycardia, or oversensing, which may have an increased incidence with extravascular implantable cardioverter defibrillators (EV-ICDs). Although other arrhythmias are dangerous and can progress into VT and VF, it is possible that other interventions such as anti-tachy pacing, or patient interventions, such as cessation of activity or mindfulness exercises, may also be efficacious at terminating the arrhythmia or the noise. Implantable cardio defibrillators (ICDs) can treat tachyarrhythmias using high-voltage defibrillation therapy, which may be referred to herein as a shock. Not all detected tachyarrhythmias, however, necessarily require a shock, and inappropriate shocks can take a significant emotional toll on ICD patients.

Avoidance of certain activities has been noted in some ICD patients as a mechanism for preventing tachyarrhythmias or causing tachyarrhythmias to cease. The systems and techniques described in this disclosure include having the patient participate in the triage and treatment planning while the tachyarrhythmia is in-progress. Including the patient in the treatment planning may reduce the number of inappropriate shocks and provide meaningful data to improve algorithms for determining when to deliver a shock.

An example system of the present disclosure includes an implanted medical device (IMD), such as an ICD, and an external device in communication with the IMD. The external device may be any of a smart phone, a tablet computer, a smart watch, a patient controller, or any other such device capable of communicating with an IMD. According to one example of the techniques described herein, an IMD may detect an arrhythmia in a patient and send to the external device an indication that the IMD has detected the arrhythmia in the patient. In some examples, the IMD establishes a secure telemetry session with the external device after an arrhythmia episode is detected to facilitate the transmission of data/commands. In response to receiving the indication from the IMD, the external device then generates an output indicating that the IMD has detected the arrhythmia. The output may, for example, be any sort of audio, visual, or haptic signal to alert a user of the external device that the IMD has detected the arrhythmia.

The external device may then solicit input from the user regarding a therapy plan for the arrhythmia. For instance, the user may be able to indicate that the patient is ready to receive a shock or that the patient wishes to delay therapy and wait to see if the arrhythmia resolves without therapy. In some examples, the patient may select an alternative therapy, such as a lower voltage shock or pacing therapy. In response to the input from the user, the external device transmits to the IMD, a command to cause the IMD to modify a therapy plan that is pre-determined by the IMD. The modification to the therapy plan may include, for example, delaying delivery of the shock to the patient, speeding up the delivery of the shock to the patient, or delivering the alternative therapy. The modification to the therapy plan may include either or both of modifying the therapy plan for the current arrhythmia or modifying the therapy plan for future arrhythmias of the same or a similar type. In some implementations, the external device may additionally recommend actions, such as cessation of activity or breathing exercises, for the patient to take to reduce the likelihood of needing a shock.

In one example implementation, the IMD may set an initial therapy plan in response to detecting the arrhythmia and then modify the initial therapy plan based on input received from the user via the external device. For instance, in response to detecting the arrhythmia the IMD may start an initial counter to set a time for delivering a shock to the patient. The initial counter may, for instance, be 30 seconds or any other predetermined amount of time. In response to receiving, via the external device, input from the user indicating that the patient is ready to receive a shock, then the IMD may deliver the shock before 30 seconds. In response to receiving, via the external device, input from the user indicating that the patient wishes to wait to see if the arrhythmia resolves without a shock, the IMD may extend the counter to 90 seconds (or any other duration of time) and deliver the shock after 90 seconds, or if the arrhythmia resolves within the 90 seconds, not deliver any shock. In scenarios where the IMD cannot establish a connection to the external device or where no user input is received, then the IMD may deliver the shock after the original counter reaches 30 seconds.

Although it is contemplated that in many use cases the user of the external device and the patient with the IMD will be the same person, it is not necessarily required that the user and the patient be the same person. For example, the user could be a caregiver or family member of the patient.

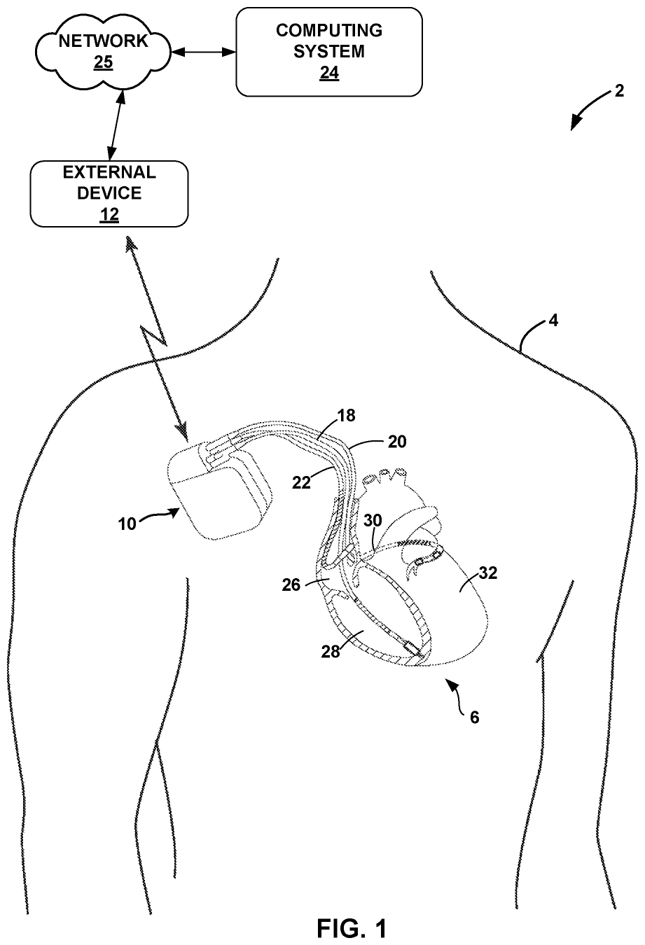

FIG. 1 is a block diagram illustrating an example system for predicting and treating cardiac arrhythmia in accordance with the techniques of the disclosure. System 2 includes a medical device. One example of such a medical device is IMD 10 depicted in FIG. 1. As illustrated by example system 10 in FIG. 1, IMD 10 may, in some examples, be an implantable cardiac pacemaker, implantable cardioverter/defibrillator (ICD), or pacemaker/cardioverter/defibrillator, for example. IMD 10 is connected to leads 18, 20 and 22 and is communicatively coupled to external device 12, which in turn is communicatively coupled to computing device 24 over communication network 25.

IMD 10 senses electrical signals attendant to the depolarization and repolarization of heart 6, e.g., a cardiac electrogram (EGM), via electrodes on one or more leads 18, 20 and 22 or the housing of IMD 10. IMD 10 may also deliver therapy in the form of electrical signals to heart 6 via electrodes located on one or more of leads 18, 20 and 22 or a housing of IMD 10. The therapy may be pacing, cardioversion and/or defibrillation pulses. IMD 10 may monitor EGM signals collected by electrodes on leads 18, 20 or 22, and based on the EGM. signal, diagnose, and treat cardiac episodes.

Leads 18, 20, 22 extend into the heart 6 of patient 4 to sense electrical activity of heart 6 and/or deliver electrical stimulation to heart 6. In the example shown in FIG. 1, right ventricular (RV) lead 18 extends through one or more veins (not shown), the superior vena cava (not shown), and right atrium 26, and into right ventricle 28. Left ventricular (LV) lead 20 extends through one or more veins, the vena cava, right atrium 26, and into the coronary sinus 30 to a region adjacent to the free wall of left ventricle 32 of heart 6. Right atrial (RA) lead 22 extends through one or more veins and the vena cava, and into the right atrium 26 of heart 12. The techniques of this disclosure may also be performed by other types of ICDs, such as leadless ICDs that do not use transvenous leads.

IMD 10 includes a plurality of electrodes (not shown in FIG. 1) and is configured to sense a cardiac EGM via the plurality of electrodes. Some or all of these electrodes may, for example, be located on leads 18, 20, and 22. IMD 10 may also include additional sensors, such as optical and impedance sensors. IMD 10 may be configured to detect an arrhythmia in patient 4. In this context, in addition to detecting active arrhythmias, detecting an arrhythmia may also include detecting the pending onset of an arrhythmia or determining that patient 4 is at an elevated risk for the onset of an arrhythmia.

External device 12 is a computing device configured for wireless communication with IMD 10. External device 12 may be configured to communicate with computing system 24 via network 25. In some examples, external device 12 may provide a user interface and allow a user to interact with IMD 10. Computing system 24 may comprise computing devices configured to allow a user to interact with IMD 10, or data collected from IMD, via network 25.

External device 12 may be used to retrieve data from IMD 10 and may transmit the data to computing system 24 via network 25. The retrieved data may include values of physiological parameters measured by IMD 10, indications of episodes of arrhythmia or other maladies detected by IMD 10, episode data collected for episodes, and other physiological signals recorded by IMD 10. The episode data may include EGM segments recorded by IMD 10, e.g., due to IMD 10 determining that an episode of arrhythmia or another malady occurred during the segment, or in response to a request to record the segment from patient 4 or another user.

In some examples, computing system 24 includes one or more handheld computing devices, computer workstations, servers or other networked computing devices. In some examples, computing system 24 may include one or more devices, including processing circuitry and storage devices. Computing system 24 and network 25 may be implemented, fully or partially, by the Medtronic Carelink™ Network or other patient monitoring systems.

Network 25 may include one or more computing devices (not shown), such as one or more non-edge switches, routers, hubs, gateways, security devices such as firewalls, intrusion detection, and/or intrusion prevention devices, servers, computer terminals, laptops, printers, databases, wireless mobile devices such as cellular phones or personal digital assistants, wireless access points, bridges, cable modems, application accelerators, or other network devices. Network 25 may include one or more networks administered by service providers, and may thus form part of a large-scale public network infrastructure, e.g., the Internet. Network 25 may provide computing devices, such as computing system 24 and IMD 10, access to the Internet, and may provide a communication framework that allows the computing devices to communicate with one another. In some examples, network 25 may be a private network that provides a communication framework that allows computing system 24, IMD 10, and/or external device 12 to communicate with one another but isolates one or more of computing system 24, IMD 10, or external device 12 from devices external to network 25 for security purposes. In some examples, the communications between computing system 24, IMD 10, and external device 12 are encrypted.

As discussed in more detail below, IMD 10 may be configured to detect an arrhythmia, and in response to detecting the arrhythmia, set an initial therapy plan for patient 4. The initial therapy plan may, for example, be that IMD will wait 45 seconds to see if the arrhythmia resolves, and if the arrhythmia has not resolved, deliver a high-voltage shock to patient 4. IMD 10 may also, either after the end of the detection interval or during the detection interval, attempt to establish a communication session with external device 12, and if a communication session can be established, transmit to external device 12 an indication that IMD 10 has detected the arrhythmia in patient 4. In some examples, the communication session is a secure telemetry session. External device 12 may be configured to receive the indication that IMD 10 has detected the arrhythmia in patient 4, e.g., via a wireless communication from IMD 10, and in response, generate an output. The output may, for example, solicit one or more inputs from patient 4, or from another user of external device 12. Based on the inputs received from patient 4, external device 12 may transmit to IMD 10 a command to cause IMD 10 to modify the initial therapy plan.

Examples of the one or more inputs that external device 12 may solicit from patient 4 include allowing a user to select between a first option to wait to see if the arrhythmia resolves without therapy or a second option to have the therapy delivered immediately. In some examples, external device 12 may also present patient 4 with an option to receive an alternative, typically a milder, therapy. In response to patient 4 selecting the first option, then external device 12 may transmit to IMD 10 a command to cause IMD 10 to increase the amount of time IMD 10 will wait before delivering a shock to patient 4. In response to patient 4 selecting the first option, computing system 10 may additionally output to patient 4 recommended actions, such as ceasing any high intensity activities, sitting down, or paced breathing. In response to patient 4 selecting the second option, then external device 12 may transmit to IMD 10 a command to cause IMD 10 to increase the amount of time IMD 10 will wait before delivering a shock to patient 4 (e.g., increase the time from 30 seconds to 90 seconds or other predetermined amount of time). In response to patient 4 selecting the third option, then external device 12 may transmit to IMD 10 a command to cause IMD 10 to deliver the alternate therapy. In response to external device 12 not receiving any input from patient 4, then external device 12 may cause IMD 10 deliver the initial therapy plan.

In one example implementation, external device 12 may be configured to present the some or all of the options described above in response to IMD 10 detecting an arrhythmia. In some implementations, external device 12 may first solicit patient status inputs from patient 4 that allow patient 4 to enter information regarding their present status. Based on these patient status inputs, external device 12 may present patient 4 with an option to defer a shock or receive an alternate therapy only if deemed appropriate based on the patient status. In some implementations, external device 12 may solicit status inputs from patient 4 after patient 4 selects the first option to defer therapy or the third option to receive an alternative therapy, and then based on the status inputs, determine an appropriate amount of time to delay therapy or an appropriate alternative therapy based on a determination of how serious a threat the arrhythmia poses. In yet other implementations, patient 4 may not make an explicit selection of whether or not to defer therapy or receive alternative therapy, but instead, external device 12 may make that determination based on the status inputs.

In some examples, the options presented to patient 4 by external device 12 may be dependent on a severity of the arrhythmia detected by IMD 10. For example, external device 12 may not present the option to defer therapy or the option to receive alternative therapy if an arrhythmia is deemed to be imminently dangerous to patient 4, or external device 12 may not present an to receive alternative therapy if the arrhythmia is determined to be of a type that would likely not respond to the alternative therapy.

Examples of the one or more status inputs that external device 12 may solicit from patient 4 include information such as an indication of how active patient 4 has recently been. For example, patient 4 may provide an input from 1 to 5, 1 to 10, or a selection of options such as low, normal, or high to indicate a recent activity level. Another example of a status input that external device 12 may solicit from patient 4 includes an indication of whether or not patient 4 has missed a dose of any medications that patient 4 regularly takes. For example, during a setup phase, patient 4 may store a list of one or more medications that patient 4 routinely takes or types or purposes of medications that patient 4 routinely takes. Then in response to IMD 10 detecting an arrhythmia, external device 12 may ask patient 4 to confirm that they have not missed any doses of these medications. Another example of a status input that external device 12 may solicit from patient 4 includes information regarding whether patient 4 has recently consumed drugs or alcohol.

In one particular example, in response to IMD 10 detecting an arrhythmia in patient 4, external device 12 may solicit from patient 4 an indication of how active patient 4 has been. As high levels of activity can be expected to naturally cause a significant increase in the heart rate of patient 4 and increase the likelihood of IMD 10 detecting a false positive, external device 12 may, without any other input from patient 4, defer therapy as long as the heart rhythm of patient 4 appears to be improving or not getting worse. If, however, the heart rhythm of patient 4 does not improve within a certain amount of time, then external device 12 may present to patient 4 an option to receive a high-voltage shock immediately, receive an alternative therapy, or continue to defer therapy. If after a certain number of deferrals, the heart rhythm of patient 4 has still not sufficiently improved, then external device 12 may stop providing patient 4 with an option to defer and alert patient 4 that they need to prepare to receive a shock.

In another example, in response to IMD 10 detecting an arrhythmia in patient 4, external device 12 may solicit from patient 4 an indication of how active patient 4 has been (e.g., prompted to confirm whether patient 4 is exercising and/or at what intensity), and patient 4 may indicate that they have been moderately active. In the absence of significant activity, patient 4 would typically be less likely to have an increased heart rate, and IMD 10 may be less likely to detect a false positive. Thus, external device 12 may be configured to not present patient 4 with an option to defer therapy or reduce the amount of time or number of times patient 4 can defer when compared to patient 4 indicating high activity.

In another example, in response to IMD 10 detecting an arrhythmia in patient 4, external device 12 may solicit from patient 4 an indication of whether or not patient 4 has missed a dose of a heart rhythm medication. As patient 4 taking their heart rhythm medication may increase the likelihood that the detected arrhythmia is a false positive or will resolve without therapy, then external device 12 may present patient 4 with an option to defer therapy for a certain amount of time. If, however, patient 4 indicates that they have missed a dose of their heart rhythm medication, then external device 12 may not present patient 4 with an option to defer therapy for a certain amount of time, but instead provide a notification to patient 4 to prepare for a shock. In other examples, if patient 4 indicates that they have missed a dose of their heart rhythm medication, then external device 12 may still present patient 4 with an option to defer therapy but for a smaller amount of time then if patient 4 had taken their medication. In some examples, the number of times that patient 4 is allowed to defer therapy may be lower if patient 4 indicates they have missed a dose of their medication compared to not missing a dose.

In another example, in response to IMD 10 detecting an arrhythmia in patient 4, external device 12 may solicit from patient 4 an indication of whether or not patient 4 has recently taken medication, drugs or alcohol. If patient 4 confirms they have taken medication, drugs or alcohol, then external device may solicit additional input regarding amounts and types of drugs or alcohol. As certain medication, drugs, or combinations can significantly increase the likelihood of life threatening arrhythmias, if patient 4 indicates they have recently taken a drug, such as a stimulant, then external device 12 not present patient 4 with an option to defer therapy or receive an alternative therapy, but instead may notify patient 4 that they need to prepare to be shocked.

The various status inputs described above may be used in any combinations or permutations to determine the various options presented by external device 12 to patient 4. As one example, if patient 4 indicates that they have not missed any medications, been moderately active, and have not consumed any medication, drugs or alcohol, then external device 12 may allow patient 4 to defer therapy an indefinite number of times as long as the severity of the detected arrhythmia is not worsening and may present patient 4 with an option to try an alternative therapy. If, however, patient 4 indicates that they have missed medications or have consumed drugs, then external device 12 may not present patient 4 with an option for alternative therapy and may only allow patient 4 to defer therapy a limited number of times before delivering a shock.

In another example, when detecting an arrhythmia in patient 4, external device 12 may present to patient 4 an option to defer a shock dependent on a detected activity level of patient 4. The activity level may, for example, be detected by accelerometers in IMD 10 or in other medical devices, such as other implantable medical devices, wearable devices, external device 12, another user device, or other such device. In response to detecting the arrhythmia in patient 4 and detecting the presence of activity, external device 12 may additionally present to patient 4 a notification to reduce or cease activity.

In another example, when detecting an arrhythmia in patient 4, external device 12 may present to patient 4 an option to defer a shock dependent on a detected respiration level of patient 4. The respiration level may, for example, be detected by sensors in IMD 10 or in other medical devices, such as other implantable medical devices or wearable devices. In response to detecting the arrhythmia in patient 4 and detecting the presence of a high respiration level, external device 12 may additionally present to patient 4 a notification to engage in breathing exercises or otherwise reduce their respiration level.

In some examples, external device 12 may output to patient 4 a timer that shows an amount of time patient 4 has to select between the various options being presented. If patient 4 does not select an option within the allotted time, then computing system may not send any command to IMD 10, such that IMD 10 continues to follow the initial therapy plan, or external device 12 may send a message to IMD 10 indicating that patient 4 has not responded. If patient 4 opts to defer therapy, then the timer may be updated to reflect the increased amount of time resulting from the deferral. External device 12 may be configured to allow patient 4 to defer therapy multiple times, but after a certain number of deferrals or in response to determining that the arrhythmia is not ceasing, external device 12 may at some point (e.g., after a predetermined amount of time or a predetermined number of deferrals) stop providing patient 4 with an option to defer therapy.

External device 12 may be configured to store the selections made by a user and correlate those selections with outcomes for patient 4. For example, external device 12 may store an indication of whether or not an arrhythmia resolved without any therapy, resolved with an alternative therapy, or required a high-voltage shock. External device 12 may then customize the options presented based on these outcomes. As one example, if for certain types of arrhythmias, alternative therapy proves unsuccessful in resolving the arrhythmia, then external device 12 may stop presenting alternative therapy as an option to patient 12. As another example, based on the stored outcomes, external device 12 may determine that for patient 4 that high activity typically corresponds to false positives or heart rates that resolve without interventions, whereas arrhythmias detected at moderate activity level tend to not resolve without therapy. External device 12 may then customize options provided to a user accordingly.

With respect to customizing options based on outcomes, such functionality may be implemented by computing system 24 instead of, or in conjunction with, external device 12. Moreover, computing system 24 may store such information for a large set of patients, and that data may be used to update the determinations made by external device 12. In some cases, external device 12 may be configured to query computing system 24 in real-time, e.g., while an arrhythmia is occurring, and computing system 24 may provide to external device 12 recommended options to present to a user. Computing system 24 may determine the recommended options based on a determined arrhythmia type, as well as information provided by a user of external device 12 regarding a status of patient 4. External device 12 may also be configured to notify computing system 24 of a number of times patient 4 has deferred therapy, so that a clinician may determine if parameters of ICD 10 need to be modified or if a treatment plan for patient 4 needs to be otherwise modified.

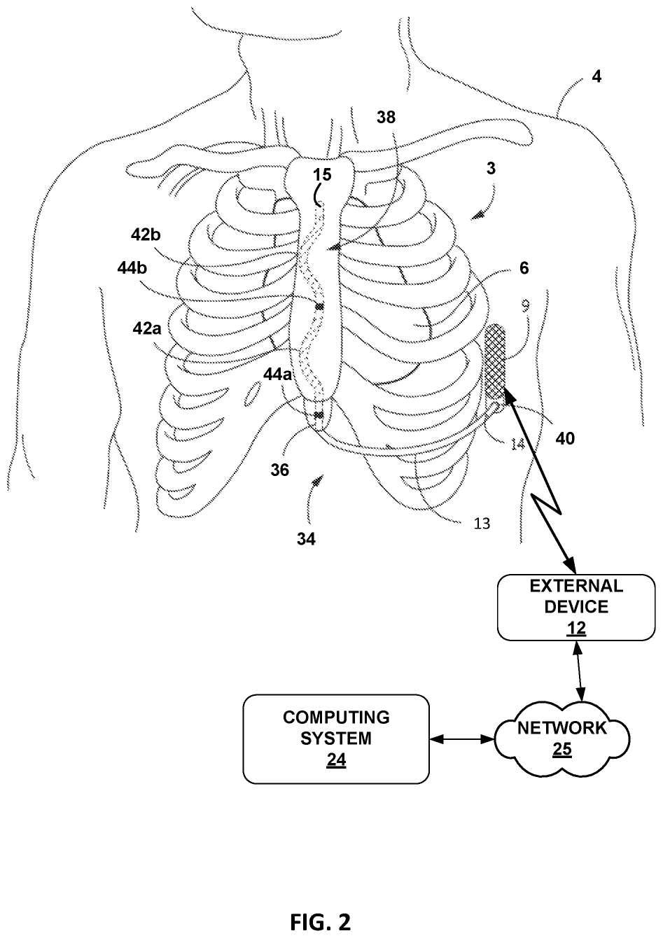

FIG. 2 is a front view of a patient 4 implanted with an extracardiovascular ICD system 3 implanted intra-thoracically. ICD system 3 includes an ICD 9 connected to an implantable medical lead 34. ICD 9 may interact with external device 12 in the same manner as ICD 10 described above. That is, ICD 9 may be configured to detect an arrhythmia, and in response to detecting the arrhythmia, set an initial therapy plan for patient 4. The initial therapy plan may, for example, be that ICD 9 will wait 45 seconds to see if the arrhythmia resolves, and if the arrhythmia has not resolved, deliver a high-voltage shock to patient 4. ICD 9 may also attempt to establish a communication session with external device 12, and if a communication session can be established, transmit to external device 12 an indication that ICD 9 has detected the arrhythmia in patient 4. In some examples, the communication session is a secure telemetry session. External device 12 may be configured to receive the indication that ICD 9 has detected the arrhythmia in patient 4, e.g., via a wireless communication from ICD 9, and in response, generate an output. The output may, for example, solicit one or more inputs from patient 4, or from another user of external device 12. Based on the inputs received from patient 4, external device 12 may transmit to ICD 9 a command to cause ICD 9 to modify the initial therapy plan.

ICD 9 may include a housing that forms a hermetic seal that protects components of the ICD 9. The housing of ICD 9 may be formed of a conductive material, such as titanium or titanium alloy, which may function as a housing electrode (sometimes referred to as a can electrode). In some embodiments, ICD 9 may be formed to have or may include a plurality of electrodes on the housing. ICD 9 may also include a connector assembly (also referred to as a connector block or header) that includes electrical feedthroughs through which electrical connections are made between conductors of lead 34 and electronic components included within the housing of ICD 9. As will be described in further detail herein, the housing may house one or more processors, memories, transmitters, receivers, sensors, sensing circuitry, therapy circuitry, power sources and other appropriate components. The housing is configured to be implanted in a patient, such patient 4.

ICD 9 is implanted extra-thoracically on the left side of patient 4, e.g., under the skin and outside the ribcage (subcutaneously or submuscularly). ICD 9 may, in some instances, be implanted between the left posterior axillary line and the left anterior axillary line of the patient. ICD 9 may, however, be implanted at other extra-thoracic locations on the patient as described later.

Lead 34 may include an elongated lead body 13 having a distal portion 15 sized to be implanted in an extracardiovascular location proximate the heart, e.g., intra-thoracically. For example, lead 34 may extend extra-thoracically under the skin and outside the ribcage (e.g., subcutaneously or submuscularly) from ICD 9 toward the center of the torso of patient 4, for example, toward the xiphoid process of patient 4. At a position proximate the xiphoid process, the lead body 13 may bend or otherwise turn and extend superiorly. The bend may be pre-formed and/or lead body 13 may be flexible to facilitate bending. In the example illustrated in FIG. 2, the lead body 13 extends superiorly intra-thoracically underneath the sternum, in a direction substantially parallel to the sternum.

Lead body 13 may extend superiorly extra-thoracically (instead of intra-thoracically), e.g., either subcutaneously or submuscularly above the ribcage/sternum. Lead 34 may be implanted at other locations, such as over the sternum, offset to the right of the sternum, angled lateral from the proximal or distal end of the sternum, or the like. In some examples, lead 34 may be implanted within an extracardiac vessel within the thorax, such as the ITV, the intercostal veins, the superior epigastric vein, or the azygos, hemiazygos, and accessory hemiazygos veins. In some examples, the distal portion 15 of lead 34 may be oriented differently than is illustrated in FIG. 2, such as orthogonal or otherwise transverse to the sternum 38 and/or inferior to heart 6. In such examples, the distal portion 15 of lead 34 may be at least partially within the anterior mediastinum of patient 4. In some examples, the distal portion 15 of lead 34 may be placed between the heart and lung as well as within the pleural cavity.

Lead body 13 may include a proximal end 15 and a distal portion 15 which include electrodes configured to deliver electrical energy to the heart or sense electrical signals of the heart. Distal portion 15 may be anchored to a desired position within the patient, for example, substernally or subcutaneously by, for example, suturing distal portion 15 to the patient's musculature, tissue, or bone at the xiphoid process entry site. In some examples, distal portion 15 may be anchored to the patient or through the use of rigid tines, prongs, barbs, clips, screws, and/or other projecting elements or flanges, disks, pliant tines, flaps, porous structures such as a mesh-like elements and metallic or non-metallic scaffolds that facilitate tissue growth for engagement, bio-adhesive surfaces, and/or any other non-piercing elements.

Lead body 13 may define a substantially linear portion 36 as it curves or bends near the xiphoid process 23 and extends superiorly. As shown in FIG. 2, at least a part of distal portion 15 may define an undulating configuration distal to the substantially linear portion 36. In particular, distal portion 15 may define an undulating pattern, e.g., zig-zag, meandering, sinusoidal, serpentine, or other pattern, as it extends toward the distal end of lead 34. In other configurations, lead body 13 may not have a substantially linear portion 36 as it extends superiorly, but instead the undulating configuration may begin immediately after the bend.

Distal portion 15 includes one or more defibrillation electrodes configured to deliver an anti-tachyarrhythmia, e.g., cardioversion/defibrillation, shock to heart 6 of patient 4. In some examples, distal portion 15 includes a plurality of defibrillation electrodes spaced a distance apart from each other along the length of distal portion 15. In the example illustrated by FIG. 2, distal portion 15 includes two defibrillation electrodes 42a and 42b (collectively, “defibrillation electrodes 42”).

Defibrillation electrodes 42 may be disposed around or within the lead body 13 of the distal portion 15, or alternatively, may be embedded within the wall of the lead body 13. In one configuration, defibrillation electrodes 42 may be coil electrodes formed by a conductor. The conductor may be formed of one or more conductive polymers, ceramics, metal-polymer composites, semiconductors, metals or metal alloys, including but not limited to, one of a combination of the platinum, tantalum, titanium, niobium, zirconium, ruthenium, indium, gold, palladium, iron, zinc, silver, nickel, aluminum, molybdenum, stainless steel, MP35N, carbon, copper, polyaniline, polypyrrole, and other polymers. In another configuration, each of defibrillation electrodes 42 may be a flat ribbon electrode, a paddle electrode, a braided or woven electrode, a mesh electrode, a directional electrode, a patch electrode or another type of electrode configured to deliver a cardioversion/defibrillation shock to heart 6 of patient 4.

Defibrillation electrodes 42 may be electrically connected to one or more conductors, which may be disposed in the body wall of lead body 13 or in one or more insulated lumens (not shown) defined by lead body 13. In an example configuration, each of defibrillation electrodes 42 is connected to a common conductor such that a voltage may be applied simultaneously to all defibrillation electrodes 42 to deliver an anti-tachyarrhythmia shock to heart 6. In other configurations, defibrillation electrodes 28 may be attached to separate conductors such that each defibrillation electrode 42 may apply a voltage independent of the other defibrillation electrodes 42. In this case, ICD 9 or lead 34 may include one or more switches or other mechanisms to electrically connect the defibrillation electrodes together to function as a common polarity electrode such that a voltage may be applied simultaneously to all defibrillation electrodes 42 in addition to being able to independently apply a voltage.

Distal portion 15 may also include one or more pacing and/or sensing electrodes configured to deliver pacing pulses to heart 6 and/or sense electrical activity of heart 6. Such electrodes may be referred to as pacing electrodes, sensing electrodes, or pace/sense electrodes. In the example illustrated by FIG. 2, distal portion 15 includes two pace/sense electrodes 44a and 44b (collectively, “pace/sense electrodes 44”).

In the example of FIG. 2, pace/sense electrode 44b is positioned between defibrillation electrodes 42, e.g., within a gap between the defibrillation electrodes, and pace/sense electrode 44a is positioned more proximal along distal portion 15 than proximal defibrillation electrode 42a. In some examples, more than one electrode 44 may exist within the gap between defibrillation electrodes 42. In some examples, an electrode 44 is additionally or alternatively located distal of the distal most defibrillation electrode 42b.

Electrodes 44 may be configured to deliver low-voltage electrical pulses to the heart or may sense a cardiac electrical activity, e.g., depolarization and repolarization of the heart. As such, electrodes 44 may be referred to herein as pace/sense electrodes 44. In one configuration, electrodes 44 are ring electrodes. However, in other configurations electrodes 44 may be any of a number of different types of electrodes, including ring electrodes, short coil electrodes, paddle electrodes, hemispherical electrodes, or directional electrodes. Each of electrodes 44 may be the same or different types of electrodes as others of electrodes 44. Electrodes 44 may be electrically isolated from an adjacent defibrillation electrode 42 by including an electrically insulating layer of material between electrodes 44 and adjacent defibrillation electrodes 42. Each electrode 44 may have its own separate conductor such that a voltage may be applied to or sensed via each electrode independently from another electrode 44.

Electrodes 42 are referred to as defibrillation electrodes, and electrodes 44 are referred to as pace/sense electrodes, because they may have different physical structures enabling different functionality. Defibrillation electrodes 42 may be larger, e.g., have greater surface area, than pace/sense electrodes 44 and, consequently, may be configured to deliver anti-tachyarrhythmia shocks that have relatively higher voltages than pacing pulses. The relatively smaller size of pace/sense electrodes 44 may provide advantages over defibrillation electrodes for delivering pacing pulses and sensing intrinsic cardiac activity, e.g., lower pacing capture thresholds and/or better sensed signal quality. Nevertheless, a defibrillation electrode 42 may be used to deliver pacing pulses and/or sense electrical activity of the heart, such as in combination with a pace/sense electrode 44.

In some examples, pace/sense electrodes 44 and the defibrillation electrodes 42 may be disposed in a common plane when distal portion 15 is implanted extracardiovasculary. In other configurations, the undulating configuration may not be substantially disposed in a common plane. For example, distal portion 15 may define a concavity or a curvature.

Proximal end 14 of lead body 13 may include one or more connectors 40 to electrically couple lead 34 to ICD 9. ICD 9 may also include a connector assembly that includes electrical feedthroughs through which electrical connections are made between the one or more connectors 40 of lead 34 and the electronic components included within the housing. The housing of ICD 9 may house one or more processors, memories, transmitters, receivers, sensors, sensing circuitry, therapy circuitry, power sources (e.g., capacitors and batteries), and/or other components. The components of ICD 9 may generate and deliver electrical therapy such as anti-tachycardia pacing, cardioversion or defibrillation shocks, post-shock pacing, and/or bradycardia pacing.

The undulating configuration of distal portion 15 and the inclusion of electrodes 44 between defibrillation electrodes 42 may provide a number of therapy vectors for the delivery of electrical therapy to the heart. For example, at least a portion of defibrillation electrodes 42 and one of electrodes 44 may be disposed over the right ventricle, or any chamber of the heart, such that pacing pulses and anti-tachyarrhythmia shocks may be delivered to the heart. The housing of ICD 9 may be charged with or function as a polarity different than the polarity of the one or more defibrillation electrodes 42 and/or electrodes 44 such that electrical energy may be delivered between the housing and the defibrillation electrode 42 and/or electrode 44 to the heart.

Each defibrillation electrode 42 may have the same polarity as every other defibrillation electrode 42 when a voltage is applied to it such that a shock may be delivered from all defibrillation electrodes together. In examples in which defibrillation electrodes 42 are electrically connected to a common conductor within lead body 13, this is the only configuration of defibrillation electrodes 42. However, in other examples, defibrillation electrodes 42 may be coupled to separate conductors within lead body 13 and may therefore each have different polarities such that electrical energy may flow between defibrillation electrodes 42, or between one of defibrillation electrodes 42 and one of pace/sense electrodes 44 or the housing electrode, to provide anti-tachyarrhythmia shock, pacing therapy, and/or to sense cardiac depolarizations. In this case, defibrillation electrodes 42 may still be electrically coupled together, e.g., via one or more switches within ICD 9, to have the same polarity.

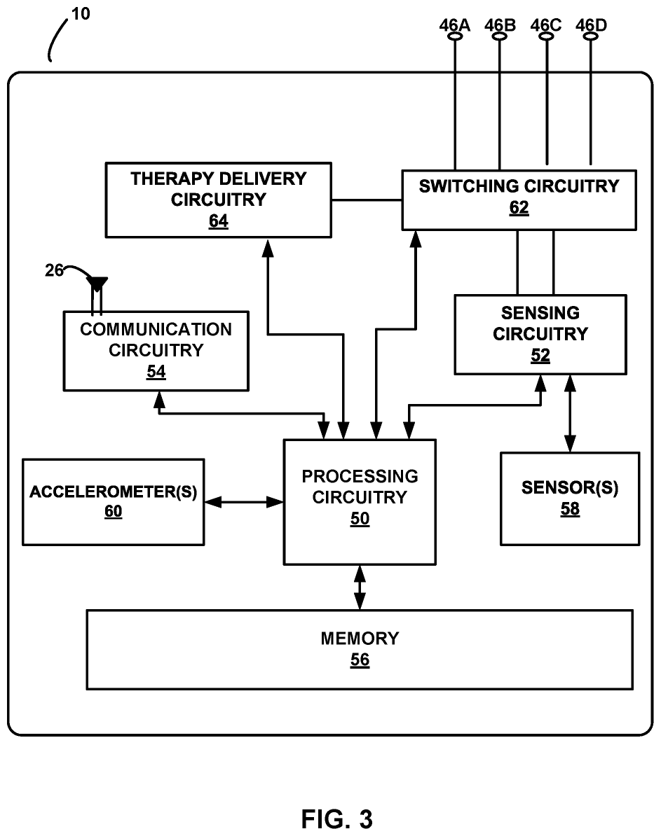

FIG. 3 is a block diagram illustrating an example configuration of IMD 10 of FIG. 1. ICD 9 of FIG. 2 may have a similar configuration. As shown in FIG. 3, IMD 10 includes processing circuitry 50, sensing circuitry 52, communication circuitry 54, memory 56, sensors 58, accelerometers 60, switching circuitry 62, therapy delivery circuitry 64, and electrodes 46A-46D (hereinafter “electrodes 46”), one or more of which may be disposed on a housing of IMD 10. In some examples, memory 56 includes computer-readable instructions that, when executed by processing circuitry 50, cause IMD 10 and processing circuitry 50 to perform various functions attributed herein to IMD 10 and processing circuitry 50. Memory 56 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to any one or more of electrodes 46 via switching circuitry 62 as controlled by processing circuitry 50. Sensing circuitry 52 may monitor signals from electrodes 46 in order to monitor electrical activity of a heart of patient 4 of FIG. 1 and produce cardiac EGM data for patient 4. In some examples, processing circuitry 50 may identify features of the sensed cardiac EGM to detect an episode of cardiac arrhythmia of patient 4. Processing circuitry 50 may store the digitized cardiac EGM and features of the EGM used to detect the arrhythmia episode in memory 56 as episode data for the detected arrhythmia episode. In some examples, processing circuitry 50 stores one or more segments of the cardiac EGM data, features derived from the cardiac EGM data, and other episode data in response to instructions from external device 12 (e.g., when patient 4 experiences one or more symptoms of arrhythmia and inputs a command to external device 12 instructing IMD 10 to upload the data for analysis by a monitoring center or clinician).

In some examples, processing circuitry 50 transmits, via communication circuitry 54, the physiological parameter data, as well as other data such as episode data, for patient 4 to an external device, such as external device 12 of FIG. 1. For example, IMD 10 sends values for various physiological parameters, digitized cardiac EGM, and other episode data to network 25 for processing by computing system 24 of FIG. 1.

Sensing circuitry 52 and/or processing circuitry 50 may be configured to detect cardiac depolarizations (e.g., P-waves of atrial depolarizations or R-waves of ventricular depolarizations) when the cardiac EGM amplitude crosses a sensing threshold. For cardiac depolarization detection, sensing circuitry 52 may include a rectifier, filter, amplifier, comparator, and/or analog-to-digital converter, in some examples. In some examples, sensing circuitry 52 may output an indication to processing circuitry 50 in response to sensing of a cardiac depolarization. In this manner, processing circuitry 50 may receive detected cardiac depolarization indicators corresponding to the occurrence of detected R-waves and P-waves in the respective chambers of heart. Processing circuitry 50 may use the indications of detected R-waves and P-waves for determining features of the cardiac EGM including inter-depolarization intervals, heart rate, and detecting arrhythmias, such as tachyarrhythmias and asystole. Sensing circuitry 52 may also provide one or more digitized cardiac EGM signals to processing circuitry 50 for analysis, e.g., for use in cardiac rhythm discrimination and/or to identify and delineate features of the cardiac EGM, such as QRS amplitudes and/or width, or other morphological features.

In some examples, IMD 10 includes one or more sensors 58, such as one or more optical sensors, impedance sensors, microphones, and/or pressure sensors. Sensors 58 may include one or more of additional sensors configured to be controlled based on detected movement, continuously-running sensors, or sensors controlled based on factors other than detected movement.

In some examples, sensing circuitry 52 may include one or more filters and amplifiers for filtering and amplifying signals received from one or more of electrodes 46 and/or other sensors 58. In some examples, sensing circuitry 52 and/or processing circuitry 50 may include a rectifier, filter and/or amplifier, a sense amplifier, comparator, and/or analog-to-digital converter. Processing circuitry 50 may determine values of physiological parameters of patient 4 based on signals from sensors 58, which may be used to identify arrhythmia episodes and stored as episode data in memory 56.

Communication circuitry 54 may include any suitable hardware, firmware, software or any combination thereof for securely communicating with another device, such as external device 12. Under the control of processing circuitry 50, communication circuitry 54 may receive downlink telemetry from, as well as send uplink telemetry to, external device 12 or another device with the aid of an internal or external antenna, e.g., antenna 26. In some examples, processing circuitry 50 may communicate with a networked computing device via an external device (e.g., external device 12) and a computer network, such as the Medtronic Carelink® Network developed by Medtronic, plc, of Dublin, Ireland.

Therapy delivery circuitry 64 may be selectively coupled to any one or more of electrodes 46 via switching circuitry 62 as controlled by processing circuitry 50. Therapy delivery circuitry 64 represents circuitry configured to deliver any of the various therapies described in this disclosure, including defibrillation therapy and pacing therapy. Therapy delivery circuitry 64 may be configured to deliver defibrillation therapy at various voltage levels ranging from, for example, 500-800 volts.

Although described herein in the context of example IMD 10, the techniques for cardiac arrhythmia detection disclosed herein may be used with other types of devices. In some examples, the functionality of IMD 10 may be implemented by separate sensing devices and therapy delivery devices. The sensing and/or therapy delivery techniques may, for example, be implemented with an extra-cardiac defibrillator coupled to electrodes outside of the cardiovascular system, a transcatheter pacemaker configured for implantation within the heart, such as the Micra™ transcatheter pacing system commercially available from Medtronic PLC of Dublin Ireland, an insertable cardiac monitor, such as the Reveal LINQ™ ICM, also commercially available from Medtronic PLC, a neurostimulator, a drug delivery device, a medical device extemal to patient 4, a wearable device such as a wearable cardioverter defibrillator, a fitness tracker, or other wearable device, a mobile device, such as a mobile phone, a “smart” phone, a laptop, a tablet computer, a personal digital assistant (PDA), or “smart” apparel such as “smart” glasses, a “smart” patch, or a “smart” watch.

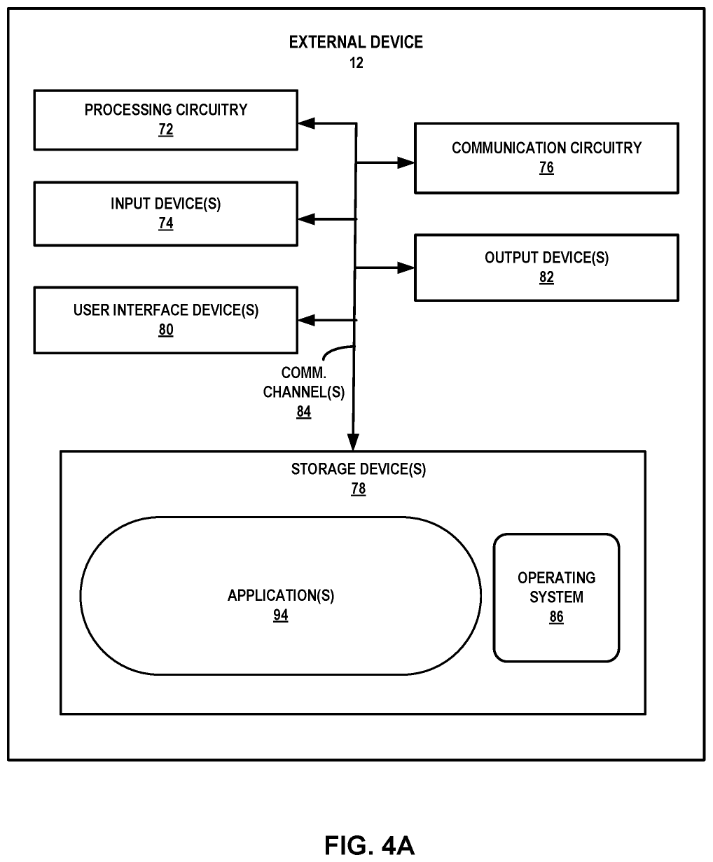

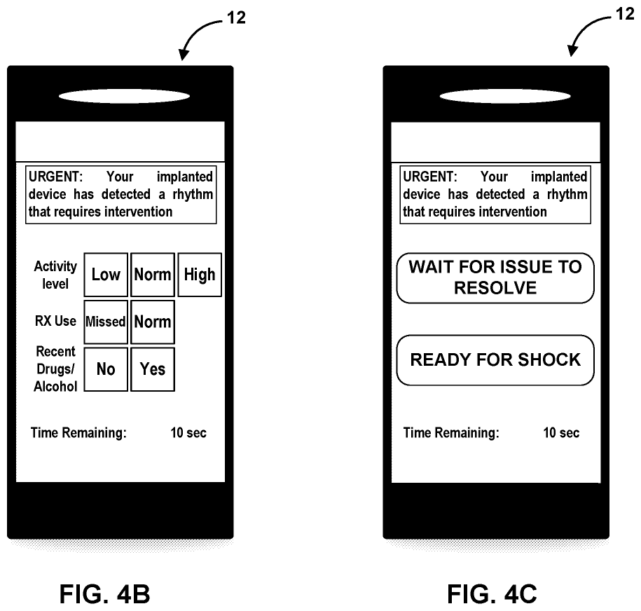

FIG. 4A is a block diagram illustrating an example configuration of external device 12. In the illustrated example, external device 12 includes processing circuitry 72 for executing applications 94 which may include cardiac episode monitoring applications or any other applications. External device 12 may be any component or system that includes processing circuitry or other suitable computing environment for executing software instructions and, for example, need not necessarily include one or more elements shown in FIG. 4A (e.g., input devices 74, communication circuitry 76, user interface devices 80, or output devices 82; and in some examples components such as storage device(s) 78 may not be co-located or in the same chassis as other components). In some examples, computing system 24 may be a cloud computing system distributed across a plurality of devices that includes external device 12.

In the example of FIG. 4A, external device 12 includes processing circuitry 72, one or more input devices 74, communication circuitry 76, one or more storage devices 78, user interface (UI) device(s) 80, and one or more output devices 82. External device 12, in some examples, further includes one or more application(s) 94, and operating system 86 that are executable by external device 12. Each of components 72, 74, 76, 78, 80, and 82 may be coupled (physically, communicatively, and/or operatively) for inter-component communications. In some examples, communication channels 84 may include a system bus, a network connection, an inter-process communication data structure, or any other method for securely communicating data. As one example, components 72, 74, 76, 78, 80, and 82 may be coupled by one or more communication channels 84.

Processing circuitry 72, in one example, is configured to implement functionality and/or process instructions for execution within external device 12. For example, processing circuitry 72 may be capable of processing instructions stored in storage device 78. Examples of processing circuitry 72 may include any one or more of a microprocessor, a controller, a digital signal processor (DSP), an application specific integrated circuit (ASIC), a field-programmable gate array (FPGA), or equivalent discrete or integrated logic circuitry.

One or more storage devices 78 may be configured to store information within computing device 70 during operation. Storage device 78, in some examples, is described as a computer-readable storage medium. In some examples, storage device 78 is a temporary memory, meaning that a primary purpose of storage device 78 is not long-term storage. Storage device 78, in some examples, is described as a volatile memory, meaning that storage device 78 does not maintain stored contents when the computer is turned off. Examples of volatile memories include RAM, dynamic random access memories (DRAM), static random access memories (SRAM), and other forms of volatile memories known in the art. In some examples, storage device 78 is used to store program instructions for execution by processing circuitry 72. Storage device 78, in one example, is used by software or applications 94 running on external device 12 to temporarily store information during program execution.

Storage devices 78, in some examples, also include one or more computer-readable storage media. Storage devices 78 may be configured to store larger amounts of information than volatile memory. Storage devices 78 may further be configured for long-term storage of information. In some examples, storage devices 78 include non-volatile storage elements. Examples of such non-volatile storage elements include magnetic bard discs, optical discs, floppy discs, flash memories, or forms of electrically programmable memories (EPROM) or electrically erasable and programmable memories (EEPROM).

External device 12, in some examples, also includes communication circuitry 76 to securely communicate with other devices and systems, such as IMD 10 and external device 12 of FIG. 1. Communication circuitry 76 may include a network interface card, such as an Ethernet card, an optical transceiver, a radio frequency transceiver, or any other type of device that can send and receive information. Other examples of such network interfaces may include 3G and Wi-Fi radios.

External device 12, in one example, also includes one or more user interface devices 80. User interface devices 80, in some examples, are configured to receive input from a user through tactile, audio, or video feedback. Examples of user interface devices(s) 80 include a presence-sensitive display, a mouse, a keyboard, a voice responsive system, video camera, microphone or any other type of device for detecting a command from a user. In some examples, a presence-sensitive display includes a touch-sensitive screen.

One or more output devices 82 may also be included in external device 12. Output devices 82, in some examples, are configured to provide output to a user using tactile, audio, or video stimuli. Output devices 82, in one example, include a presence-sensitive display, a sound card, a video graphics adapter card, or any other type of device for converting a signal into an appropriate form understandable to humans or machines. Additional examples of output devices 82 include a speaker, a cathode ray tube (CRT) monitor, a liquid crystal display (LCD), or any other type of device that can generate intelligible output to a user.

External device 12 may include operating system 86. Operating system 86, in some examples, controls the operation of components of external device 12. For example, operating system 86, in one example, facilitates the communication of one or more applications 94 with processing circuitry 72, communication circuitry 76, storage device 78, input device 74, user interface devices 80, and output device 82.

Applications 94 may also include program instructions and/or data that are executable by computing device 70. Example application(s) 94 executable by computing device 70 may include cardiac monitoring applications or applications for monitoring other conditions of patient 4. Other additional applications not shown may alternatively or additionally be included to provide other functionality described herein and are not depicted for the sake of simplicity.

In accordance with the techniques of the disclosure, external device 12 receives episode data for episodes stored by medical devices, such as IMD 10, via communication circuitry 76. Storage device 78 may store the episode data for the episodes in storage device 78. The episode data may have been collected by the medical devices in response to the medical devices detecting arrhythmias and/or user input directing the storage of episode data.

FIGS. 4B and 4C show examples of external device 12 and, more particularly, show examples of displays that may be presented to a user of external device 12 in response to IMD 10 detecting an arrhythmia. In the example of FIG. 4B, a display device of external device 12 is presenting an alert indicating the IMD 10 detected a rhythm that requires intervention and is present a user with options related to activity level, medication (Rx) usage, and recent drug or alcohol usage. In the example of FIG. 4B, the display of device 12 is also presenting a timer showing that the user has 10 seconds remaining to make a selection.

In the example of FIG. 4C, a display device of external device 12 is presenting an alert indicating the IMD 10 detected a rhythm that requires intervention and is presenting a user with options wait to see if the issues resolves or for the user to indicate they are ready to receive a shock. In the example of FIG. 4C, the display of device 12 is also presenting a timer showing that the user has 10 seconds remaining to make a selection. As explained above, external device 12 may be configured to present the options of only one of either FIG. 4B or FIG. 4C, or to present the options of FIG. 4C conditionally based on responses received to the options of FIG. 4B, or vice versa.

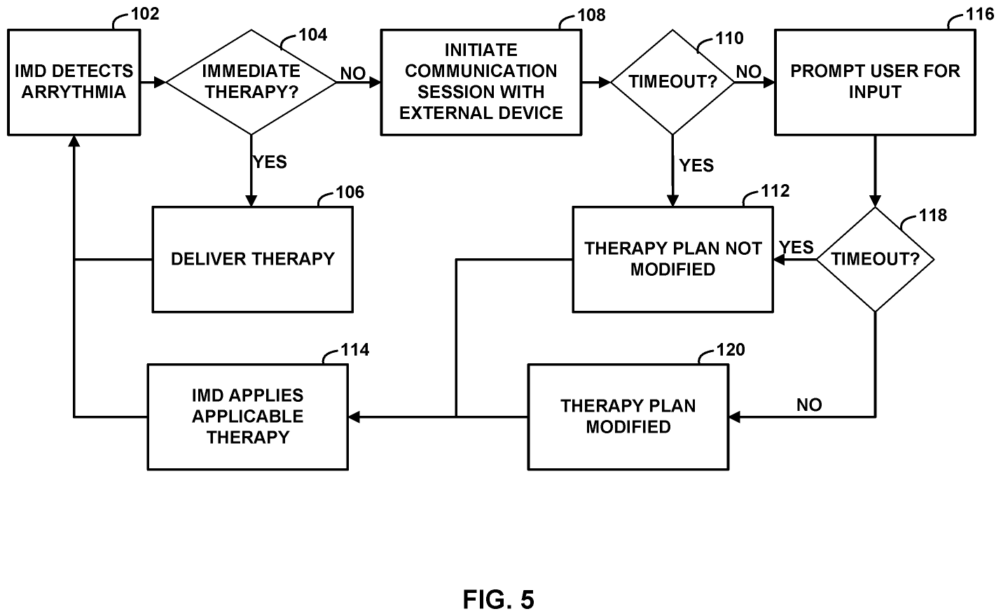

FIG. 5 shows a flow diagram of a process that may be performed by IMD 10 and external device 12. In the example of FIG. 5, IMD 10 detects an arrhythmia in patient 4 (102) and determines an initial therapy plan for patient 4. If the arrhythmia requires immediate therapy (104, YES), then IMD 10 delivers the therapy (106). When the arrhythmia requires immediate therapy, then IMD 10 may deliver the therapy without giving patient 4 an option to defer the therapy or an option to receive an alternate therapy. If the arrhythmia does not requires immediate therapy (104, NO), then IMD 10 attempts to initiate a communication session with external device 12 (108). In some examples, the attempted communication session is a secure telemetry session. If IMD 10 cannot establish a communication session with external device 12 without timing out (110, YES), then IMD 10 does not modify the initial therapy plan (112) and provides patient 4 with the applicable therapy (114), which in this scenario would be the initial therapy.

If IMD 10 can establish a communication session with external device 12 without timing out (110, NO), then IMD 10 causes external device 12 to prompt a user for input (116). The prompts and the input may take the forms and include the information described above. If the user does not respond within a certain amount of time (e.g., 30 seconds or any other predetermined amount of time) (118, YES), then external device 12 does not cause IMD to modify the initial therapy plan (112), and IMD provides patient 4 with the applicable therapy (114), which in this scenario would be either the initial therapy or a most recently modified therapy plan. If the user does respond within a certain amount of time (e.g., 30 seconds or any other predetermined amount of time) (118, NO), then external device 12 may cause IMD 10 to modify the therapy plan based on the input received from the user (120), and IMD 10 delivers the applicable therapy (114), which in this case may be a therapy plan that has been modified based on user input. The modification to the therapy plan may include either or both of modifying the therapy plan for the current arrhythmia or modifying the therapy plan for future arrhythmias of the same or a similar type. For example, IMD 10 may set the modified therapy plan to be the initial therapy plan for future arrhythmias of the same or a similar type. In some implementations, IMD 10 or user device 12 may store a plurality of user inputs or a plurality of therapy plans resulting from the user inputs and set the initial therapy plan to be the most frequently occurring therapy plan resulting from the user inputs.

In some examples, IMD 10 may perform one or more of steps 104, 108, 110, 116, 118, and 114 after step 106. That is IMD 10 may deliver a therapy (106), then prompt a user for input (116), modify a therapy plan (120) for future arrhythmias, and apply the modified therapy plan to future arrhythmias (114).

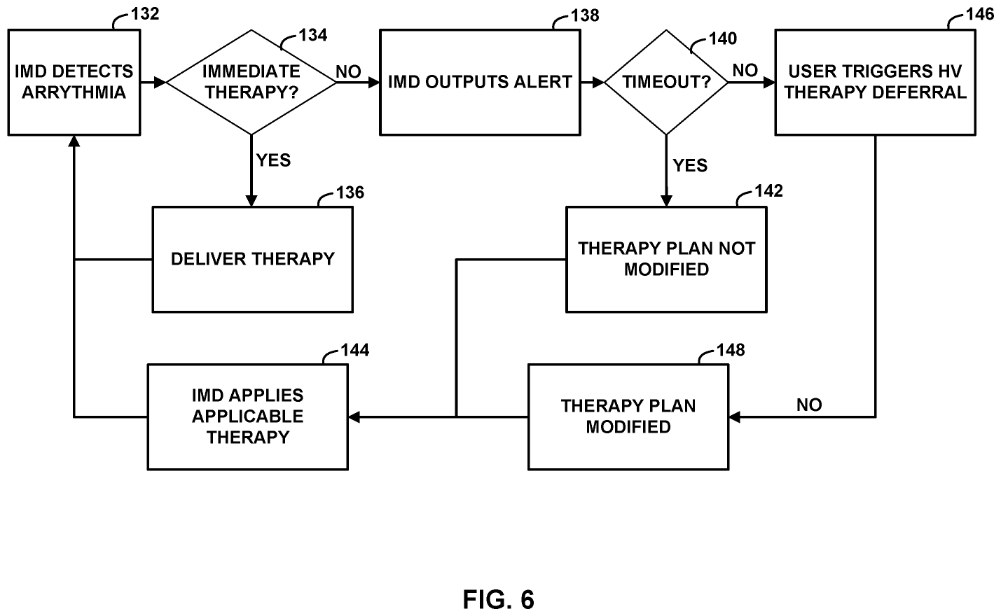

FIG. 6 shows a flow diagram of a process that may be performed by IMD 10 without external device 12. In the example of FIG. 5, IMD 10 detects an arrhythmia in patient 4 (132) and determines an initial therapy plan for patient 4. If the arrhythmia requires immediate therapy (134, YES), then IMD 10 delivers the therapy (136). When the arrhythmia requires immediate therapy, then IMD 10 may deliver the therapy without giving patient 4 an option to defer the therapy or an option to receive an alternate therapy. If the arrhythmia does not requires immediate therapy (134, NO), then IMD 10 attempts to alert patient 4 that IMD 10 has detected the arrhythmia (138). The alert may be any sort of audible or haptic alert that can attract the attention of patient 4. If IMD 10 does not, within a set amount of time (e.g., 30 seconds or any other predetermined amount of time), receive feedback from patient 4 acknowledging that IMD has detected an arrhythmia (140, YES), then IMD 10 does not modify the initial therapy plan (142) and provides patient 4 with the applicable therapy (144), which in this scenario would be the initial therapy.

If IMD 10 does receive feedback from patient 4 before timing out (e.g., within 30 seconds or any other predetermined amount of time) (140, NO), then IMD 10 may determine that patient 4 wishes to defer high-voltage therapy (146), and IMD 10 can modify the therapy plan accordingly (148). IMD 10 then applies the applicable therapy, which in this case is a modified therapy plan that may delay or defer delivery of high-voltage therapy compared to the initial therapy plan. The deferral of the high-voltage therapy may apply to either or both of the therapy plan for the current arrhythmia or therapy plans for future arrhythmias of the same or a similar type. For example, IMD 10 may set the initial therapy plan for future arrhythmias to also include deferral of high-voltage therapy.

In some examples, IMD 10 may perform one or more of steps 134, 138, 140, 146, 148, and 144 after step 136. That is IMD 10 may deliver a therapy (136), then prompt a user for input (146), modify a therapy plan (148) for future arrhythmias, and apply the modified therapy plan to future arrhythmias (144).

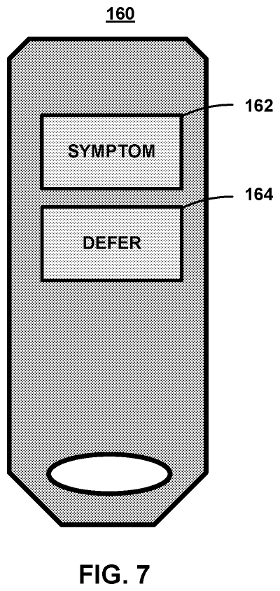

In the example of FIG. 6 which does not require an external computing device like external device 12, IMD 10 may be configured to receive the feedback or information from patient 4 in any of a variety of manners. In on example, patient 4 may have a simple electronic device that takes a form similar to car alarm remote, such as device 160 in FIG. 7. Device 160 includes two buttons but other implementations could include more or fewer buttons. In the example of FIG. 7, patient 4 may be able to press first button 162 to confirm the presence of symptoms, which may cause IMD 10 to not defer delivery of high-voltage therapy or press second button 164 to indicate that they wish to defer therapy (e.g., for a predetermined amount of time). In other implementations, patient 4 may additionally or alternatively be able to defer therapy by uttering a voice command that can be interpreted by IMD 10, touching a part of their body at a location that can be sensed by IMD 10, making a movement that can be sensed by accelerometers of IMD 10, or using any other such mechanism.

In some examples, the techniques of the disclosure include a system that comprises means to perform any method described herein. In some examples, the techniques of the disclosure include a computer-readable medium comprising instructions that cause processing circuitry to perform any method described herein.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following clauses are illustrative of the techniques and systems described herein.

Clause 1. A method comprising: receiving, at an external device from an implantable medical device (IMD) implanted in a patient, an indication that the IMD has detected an arrhythmia in the patient; generating, by the external device, an output indicating that the IMD has detected the arrhythmia; receiving, at the external device, an input from a user of the external device; and in response to the input from the user, transmitting, from the external device to the IMD, a command to cause the IMD to modify a therapy plan determined by the IMD.

Clause 2. The method of clause 1, wherein the input comprises a request by the user to delay receiving defibrillation therapy from the IMD.

Clause 3. The method of clause 1, wherein the input comprises a request by the user to expedite receiving defibrillation therapy from the IMD.

Clause 4. The method of clause 1, wherein the input comprises an indication of an activity level of the patient.

Clause 5. The method of clause 1, wherein the input comprises a confirmation from the user that the patient has taken a medication.

Clause 6. The method of any of clauses 1-5, wherein the therapy plan includes delivering defibrillation therapy at a first voltage, and the command causes the IMD to modify the therapy plan to include delivering the defibrillation therapy at a second voltage different than the first voltage.

Clause 7. The method of clause 6, wherein the command causes the IMD to modify the therapy plan to include delivering the defibrillation therapy at the second voltage different than the first voltage in response to the IMD detecting a subsequent arrhythmia in the patient.

Clause 8. The method of any of clauses 1-5, wherein the therapy plan includes delivering defibrillation therapy, and the command causes the IMD to modify the therapy plan to include pacing therapy instead of the defibrillation therapy.

Clause 9. The method of clause 8, wherein the command causes the IMD to modify the therapy plan to include delivering the pacing therapy instead of the defibrillation therapy in response to the IMD detecting a subsequent arrhythmia in the patient.

Clause 10. The method of any of clauses 1-5, wherein the therapy plan includes delivering defibrillation therapy at a first time, and the command causes the IMD to modify the therapy plan to deliver the defibrillation therapy at a second time that is later than the first time.

Clause 11. The method of clause 10, wherein the therapy plan includes delivering defibrillation therapy at the first time after detection of the arrhythmia, and the command causes the IMD to modify the therapy plan to deliver the defibrillation therapy at the second time after detection of a subsequent arrhythmia.

Clause 12. The method of any of clauses 1-11, further comprising storing a plurality of inputs received from the user in response to a plurality of outputs indicating that the IMD has detected the arrhythmia in the patient, wherein the plurality of inputs includes the input, and wherein the command causes the IMD to modify the therapy plan based on the plurality of inputs.

Clause 13. The method of clause 12, wherein the command causes the IMD to modify the therapy plan based on a most frequently occurring input of the plurality of inputs.

Clause 14. The method of any of clauses 1-13, further comprising: in response to receiving the indication that the IMD has detected the arrhythmia in the patient, establishing a secure telemetry session between the IMD and the external device to facilitation transmission of data between the IMD and the external device.

Clause 15. A device comprising: a memory; and processing circuitry coupled to the memory and configured to: receive, from an implantable medical device (IMD) implanted in a patient, an indication that the IMD has detected an arrhythmia in the patient; generate an output indicating that the IMD has detected the arrhythmia; receive, at the external device, an input from a user of the external device; and in response to the input from the user, transmit to the IMD a command to cause the IMD to modify a therapy plan determined by the IMD.

Clause 16. The device of clause 15, wherein the input comprises a request by the user to delay receiving defibrillation therapy from the IMD.

Clause 17. The device of clause 15, wherein the input comprises a request by the user to expedite receiving defibrillation therapy from the IMD.

Clause 18. The device of clause 15, wherein the input comprises an indication of an activity level of the patient.

Clause 19. The device of clause 15, wherein the input comprises a confirmation from the user that the patient has taken a medication.

Clause 20. The device of any of clauses 15-19, wherein the therapy plan includes delivering defibrillation therapy at a first voltage, and the command causes the IMD to modify the therapy plan to include delivering the defibrillation therapy at a second voltage different than the first voltage.

Clause 21. The device of clause 20, wherein the command causes the IMD to modify the therapy plan to include delivering the defibrillation therapy at the second voltage different than the first voltage in response to the IMD detecting a subsequent arrhythmia in the patient.

Clause 22. The device of any of clauses 15-19, wherein the therapy plan includes delivering defibrillation therapy, and the command causes the IMD to modify the therapy plan to include pacing therapy instead of the defibrillation therapy.

Clause 23. The device of clause 22, wherein the command causes the IMD to modify the therapy plan to include delivering the pacing therapy instead of the defibrillation therapy in response to the IMD detecting a subsequent arrhythmia in the patient.

Clause 24. The device of any of clauses 15-19, wherein the therapy plan includes delivering defibrillation therapy at a first time, and the command causes the IMD to modify the therapy plan to deliver the defibrillation therapy at a second time that is later than the first time.

Clause 25. The device of clause 24, wherein the therapy plan includes delivering defibrillation therapy at the first time after detection of the arrhythmia, and the command causes the IMD to modify the therapy plan to deliver the defibrillation therapy at the second time after detection of a subsequent arrhythmia.

Clause 26. The device of any of clauses 15-25, wherein the processing circuitry is configured to: in response to receiving the indication that the IMD has detected the arrhythmia in the patient, establish a secure telemetry session with the IMD.

Clause 27. The device of any of clauses 15-26, wherein the processing circuitry is further configured to store a plurality of inputs received from the user in response to a plurality of outputs indicating that the IMD has detected the arrhythmia in the patient, wherein the plurality of inputs includes the input, and wherein the command causes the IMD to modify the therapy plan based on the plurality of inputs.

Clause 28. The device of clause 27, wherein the command causes the IMD to modify the therapy plan based on a most frequently occurring input of the plurality of inputs.

Clause 29. A system comprising: an implantable medical device (IMD) configured to detect an arrhythmia in a patient; and an external device comprising processing circuitry and configured to: receive from the IMD an indication that the IMD has detected the arrhythmia in the patient; generate an output indicating that the IMD has detected the arrhythmia; receive an input from a user; and in response to the input from the user, transmitting to the IMD a command to cause the IMD to modify a therapy plan determined by the IMD.

Clause 30. The system of clause 29, wherein the input comprises a request by the user to delay receiving defibrillation therapy from the IMD.

Clause 31. The system of clause 29, wherein the input comprises a request by the user to expedite receiving defibrillation therapy from the IMD.

Clause 32. The system of clause 29, wherein the input comprises an indication of an activity level of the patient.

Clause 33. The system of clause 29, wherein the input comprises a confirmation from the user that the patient has taken a medication.

Clause 34. The system of any of clauses 29-34, wherein the therapy plan includes delivering defibrillation therapy at a first voltage, and the command causes the IMD to modify the therapy plan to include delivering the defibrillation therapy at a second voltage different than the first voltage.

Clause 35. The system of clause 34, wherein the command causes the IMD to modify the therapy plan to include delivering the defibrillation therapy at the second voltage different than the first voltage in response to the IMD detecting a subsequent arrhythmia in the patient.

Clause 36. The system of any of clauses 29-34, wherein the therapy plan includes delivering defibrillation therapy, and the command causes the IMD to modify the therapy plan to include pacing therapy instead of the defibrillation therapy.

Clause 37 The system of clause 36, wherein the command causes the IMD to modify the therapy plan to include delivering the pacing therapy instead of the defibrillation therapy in response to the IMD detecting a subsequent arrhythmia in the patient.

Clause 38. The system of any of clauses 29-34, wherein the therapy plan includes delivering defibrillation therapy at a first time, and the command causes the IMD to modify the therapy plan to deliver the defibrillation therapy at a second time that is later than the first time.

Clause 39. The system of clause 38, wherein the therapy plan includes delivering defibrillation therapy at the first time after detection of the arrhythmia, and the command causes the IMD to modify the therapy plan to deliver the defibrillation therapy at the second time after detection of a subsequent arrhythmia.

Clause 40. The system of any of clauses 29-39, wherein the processing circuitry is further configured to store a plurality of inputs received from the user in response to a plurality of outputs indicating that the IMD has detected the arrhythmia in the patient, wherein the plurality of inputs includes the input, and wherein the command causes the IMD to modify the therapy plan based on the plurality of inputs.

Clause 41. The system of clause 40, wherein the command causes the IMD to modify the therapy plan based on a most frequently occurring input of the plurality of inputs.

Clause 42. The system of any of clauses 29-41, wherein the IMD is configured to establish a secure telemetry session with the external device to facilitate transmission of data between the IMD and the external device in response to the IMD detecting the arrhythmia in the patient.

Clause 43. The system of any of clauses 29-42, wherein the external device is configured to establish a secure telemetry session with the IMD to facilitate transmission of data between the IMD and the external device in response to the IMD detecting the arrhythmia in the patient.

Clause 44. A computer-readable storage medium storing instructions that when executed by one or more processors cause the one or more processors to perform the method of any of clauses 1-14.

Various examples have been described. These and other examples are within the scope of the following claims.

1. A device comprising:
a memory; and
processing circuitry coupled to the memory and configured to:
receive, from an implantable medical device (IMD) implanted in a patient, an indication that the IMD has detected an arrhythmia in the patient;
generate an output indicating that the IMD has detected the arrhythmia;
receive, via an external device, an input from a user of the external device; and
in response to the input from the user, transmit to the IMD a command to cause the IMD to modify a therapy plan determined by the IMD.

2. The device of claim 1, wherein the input comprises a request by the user to delay receiving defibrillation therapy from the IMD.

3. The device of claim 1, wherein the input comprises a request by the user to expedite receiving defibrillation therapy from the IMD.

4. The device of claim 1, wherein the input comprises an indication of an activity level of the patient.

5. The device of claim 1, wherein the input comprises a confirmation from the user that the patient has taken a medication.

6. The device of claim 1, wherein the therapy plan includes delivering defibrillation therapy at a first voltage, and the command causes the IMD to modify the therapy plan to include delivering the defibrillation therapy at a second voltage different than the first voltage.

7. The device of claim 1, wherein the therapy plan includes delivering defibrillation therapy, and the command causes the IMD to modify the therapy plan to include pacing therapy instead of the defibrillation therapy.

8. The device of claim 1, wherein the therapy plan includes delivering defibrillation therapy at a first time, and the command causes the IMD to modify the therapy plan to deliver the defibrillation therapy at a second time that is later than the first time.

9. The device of claim 1, wherein the processing circuitry is configured to:
in response to receiving the indication that the IMD has detected the arrhythmia in the patient, establish a secure telemetry session with the IMD.

10. A system comprising:
an implantable medical device (IMD) configured to detect an arrhythmia in a patient; and
an external device comprising processing circuitry and configured to:
receive from the IMD an indication that the IMD has detected the arrhythmia in the patient;
generate an output indicating that the IMD has detected the arrhythmia;
receive an input from a user; and
in response to the input from the user, transmitting to the IMD a command to cause the IMD to modify a therapy plan determined by the IMD.

11. The system of claim 10, wherein the input comprises one of a request by the user to delay receiving defibrillation therapy from the IMD, a request by the user to expedite receiving defibrillation therapy from the IMD, an indication of an activity level of the patient, or a confirmation from the user that the patient has taken a medication.

12. The system of claim 10, wherein the processing circuitry is further configured to apply the modified therapy plan to a subsequent arrhythmia following the detected arrhythmia.

13. A method comprising:
receiving, at an external device from an implantable medical device (IMD) implanted in a patient, an indication that the IMD has detected an arrhythmia in the patient;
generating, by the external device, an output indicating that the IMD has detected the arrhythmia;
receiving, at the external device, an input from a user of the external device; and
in response to the input from the user, transmitting, from the external device to the IMD, a command to cause the IMD to modify a therapy plan determined by the IMD.

14. The method of claim 13, wherein the input comprises one of a request by the user to delay receiving defibrillation therapy from the IMD, a request by the user to expedite receiving defibrillation therapy from the IMD, an indication of an activity level of the patient, or a confirmation from the user that the patient has taken a medication.

15. The method of claim 13 or 14, wherein the command causes the IMD to apply the modified therapy plan to a subsequent arrhythmia following the arrhythmia.

16. The method of claim 13, wherein the therapy plan includes delivering defibrillation therapy at a first voltage, and the command causes the IMD to modify the therapy plan to include delivering the defibrillation therapy at a second voltage different than the first voltage.

17. The method of claim 13, wherein the therapy plan includes delivering defibrillation therapy, and the command causes the IMD to modify the therapy plan to include pacing therapy instead of the defibrillation therapy.

18. The system of claim 10, wherein the therapy plan includes delivering defibrillation therapy at a first voltage, and the command causes the IMD to modify the therapy plan to include delivering the defibrillation therapy at a second voltage different than the first voltage.

19. The system of claim 10, wherein the therapy plan includes delivering defibrillation therapy, and the command causes the IMD to modify the therapy plan to include pacing therapy instead of the defibrillation therapy.

20. The system of claim 10, wherein the therapy plan includes delivering defibrillation therapy at a first time, and the command causes the IMD to modify the therapy plan to deliver the defibrillation therapy at a second time that is later than the first time.
